SEQUENCE LISTING (8 sequences)

SEQ ID NO: 1 - DNA, 18 bp, Artificial Sequence
  synthetic sequence
tccccggcatcgtggcgg

SEQ ID NO: 2 - DNA, 16 bp, Artificial Sequence
  synthetic sequence
gaccttgtctgcgggc

SEQ ID NO: 3 - DNA, 21 bp, Artificial Sequence
  synthetic sequence
gtgtgttgctgcacttaattc

SEQ ID NO: 4 - DNA, 32 bp, Artificial Sequence
  synthetic sequence
gggcagcttgtcgatccccggcatcgtggcgg

SEQ ID NO: 5 - DNA, 18 bp, Artificial Sequence
  synthetic sequence
tgggcggctacgtgcgcg

SEQ ID NO: 6 - DNA, 21 bp, Artificial Sequence
  Combined DNA/RNA sequence (siRNA with thymidine tag)
gaugccacagcuauuuccann

SEQ ID NO: 7 - DNA, 21 bp, Artificial Sequence
  synthetic sequence
cagtcgcgtttgcgactggtt

SEQ ID NO: 8 - DNA, 93 bp, Artificial Sequence
  synthetic sequence
aaaaaagtccactggaagcagctaccaaccaacctcaagcttcaagtcggttgatagctgcttccagtagacggtgtttcgtcctttccacaa

(19) United States
(12) Patent Application Publication
(10) Pub. No.: US 2005/0065105 A1
(43) Pub. Date: Mar. 24, 2005

(54) Modulation of APPL expression

(76) Inventors: Joseph R. Testa, Churchville, PA (US); Yasuhiro Mitsuuchi, Jenkintown, PA (US); Poulikos I. Poulikakos, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/785,168

(22) Filed: Feb. 23, 2004

Related U.S. Application Data
(63) Continuation-in-part of application No. PCT/US02/27018, filed on Aug. 23, 2002.
(60) Provisional application No. 60/314,530, filed on Aug. 23, 2001.

(51) Int. Cl. C12Q 1/68; C07H 21/04; A61K 48/00

(52) U.S. Cl. 514/44; 435/6; 536/23.2

(57) ABSTRACT

Compositions and methods are provided for modulating the expression of APPL.

This application is a continuation in part of PCT/US02/27018, filed Aug. 23, 2002, which claims priority to U.S. Provisional Application 60/314,530 filed Aug. 23, 2001, the entire disclosure of both of these applications is incorporated by reference herein.

Pursuant to 35 U.S.C. Section 202(c), it is acknowledged that the United States Government has certain rights in the invention described herein, which was made in part with funds from the National Institutes of Health Grant No. CA77429.

### FIELD OF THE INVENTION

This invention relates to the fields of molecular biology, cell proliferation, and programmed cell death. Specifically, the invention provides oligonucleotide molecules targeted to nucleic acids encoding APPL which induce cell growth suppression and/or cell death. Methods for inhibiting the proliferation of cancer cells using the oligonucleotides of the invention alone and in combination with other anti-cancer agents are also provided.

### BACKGROUND OF THE INVENTION

Several publications and patent documents are referenced in this application by author name and year of publication in parentheses in order to more fully describe the state of the art to which this invention pertains. Full citations for these references are found at the end of the specification. The disclosure of each of these publications and patent documents is incorporated by reference herein.

AKT2 and the closely related AKT1 are human cellular homologues of the viral oncogene v-akt (Staal, 1987). The AKT2 oncogene has been shown to be amplified and/or overexpressed in human ovarian and pancreatic carcinomas (Cheng et al., 1992; Ruggeri et al., 1998). In normal adult mice, expression of Akt2 mRNA and AKT2 protein is ubiquitous, with highest levels in skeletal muscle (Altomare et al., 1995). In situ hybridization of mouse embryos demonstrated that Akt2 is expressed more abundantly than Akt (the murine homologue of human AKT1) in muscle, liver and especially brown fat—tissues which are highly insulin-responsive and actively involved in glucose metabolism (Altomare et al., 1998).

The AKT2 protein (also known as protein kinase Bb, PKBb) is a serine/threonine kinase related to protein kinases A and C (Jones et al., 1991; Cheng et al., 1992). The amino terminus of AKT2 contains a pleckstrin homology (PH) domain, a region shared by many molecules involved in signal transduction. Like AKT1, the AKT2 kinase is activated in response to a wide variety of growth factors or insulin through phosphatidylinositol 3-kinase (PI3K) (Mitsuuchi et al., 1998; Liu et al., 1998). Ras can also activate AKT2 presumably via direct interaction with the PI3K catalytic subunit, p110Δ (Liu et al., 1998). AKT2 and other members of the AKT/PKB family are activated by 3-phosphoinositide-dependent protein kinase-1 in the presence of the PI3K products phosphatidylinositol 3,4,5-trisphosphate (PtdIns(3,4,5)P3) and phosphatidylinositol 3,4-bisphosphate (PtdIns(3,4)P2) (Walker et al., 1998). Furthermore, integrin-linked kinase regulates AKT1 activity in a PI3K-dependent manner (Delcommenne et al., 1998).

Recent studies have linked AKT1 to such diverse processes as inhibition of apoptosis, by phosphorylating BAD and caspase-9 (Datta et al., 1997; del Peso et al., 1997; Blume-Jensen et al., 1998), and insulin metabolism, by regulating intracellular trafficking of vesicles containing glucose transporter 4 (GLUT4) (Cong et al., 1997). The elevated expression of AKT2 in brown fat and other tissues prominently involved in glucose metabolism (Altomare et al., 1998) suggested that AKT2 may mediate some of the effects of insulin by influencing glucose uptake via membrane translocation of GLUT4. Indeed, recently AKT2 has been observed in Glut4-containing vesicles (Calera et al., 1998). Moreover, both AKT1 and AKT2 inactivate glycogen synthase kinase-3 (GSK3), which suggested a potentially important role of AKT family members in glycogen synthesis (Cross et al., 1995; Mitsuuchi et al., 1998).

Despite the fact that AKT2 and AKT1 share certain downstream targets, these kinases differ with regard to their involvement in human cancer. Unlike AKT2, alterations of AKT1 are rarely reported in human cancers, and AKT2, but not AKT1, has been shown to be oncogenic when experimentally overexpressed in mammalian cells (Cheng et al., 1997).

In order to further characterize the molecular role of these Akt family kinases, yeast two-hybrid screening was performed using human AKT2 as bait. These experiments identified a novel protein containing multiple signaling motifs. This AKT2 partner, designated APPL, is an Adaptor protein containing a Pleckstrin homology domain, a Phosphotyrosine binding (PTB) domain, and a Leucine zipper motif.

APPL encodes predicted coiled-coil structures at its amino and carboxyl termini. Secondary structure and coiled-coil structure predictions indicate that the carboxyl region contains a single helical bundle similar to those reported for centromeric protein E, membrane-organizing extension spike protein, and troponin T. The PH domain consists of approximately 100 amino acids, a typical size for such domains. A similarity search revealed that this sequence is similar to the PH domains of oligophrenin-1 and Graf, two members of the GTPase-activating protein (GAP) family. The PTB domain of APPL shares 47% similarity with the PTB domains of both CED-6, an adaptor protein that promotes engulfment of apoptotic cells in C. elegans, and IB1, a GLUT2 gene transactivator.

APPL is highly expressed in skeletal muscle, heart, ovary, and pancreas—tissues in which Akt2 mRNA is abundant. Therefore, APPL's involvement in AKT2 signaling represents an important target for the treatment of disease entities, such as cancer, in which AKT2 has been implicated in their pathology.

APPL has also been shown to interact with the catalytic subunit of phosphatidylinositol 3-kinase, p110Δ. Thus, APPL appears to act as an adaptor that tethers inactive AKT2 to p110 Δ in the cytoplasm, thereby expediting recruitment of these molecules to the cell membrane upon mitogenic stimulation.

The APPL-AKT2 interaction occurs between the PTB domain of APPL and the catalytic domain of AKT2. The PTB domain of Shc recognizes phosphorylated tyrosine residues within the consensus sequence fNPXphosphoY (where f signifies a hydrophobic amino acid, X refers to any amino acid, and N, P, and Y represent Asn, Pro, and Tyr, respectively; SEQ ID NO: 6) (Kavanaugh et al., 1995; Zhou et al., 1995). However, AKT2 does not possess this consensus sequence, suggesting that the APPL(PTB)-AKT2 interaction differs from that occurring between the PTB domain of Shc and its target proteins.

APPL has also been shown to interact with the actin cytoskeleton machinery. Specifically, APPL has been observed at punctate spots along actin filaments, especially in motile regions known as lamellipodia. Moreover, the accumulation noted at lamellipodia has been determined to be mediated through Rac1 signaling. Rac1 is a member of the Rho guanosine triphosphatases (GTPases) which are known to coordinate many diverse cellular processes such as adhesion, migration, cyokinesis, cell cycle progression, and proliferation. Additionally, Rho GTPases have been implicated in tumor cell invasion and in some aspects of metastasis.

APPL has been mapped to the 3p14.3-21.1 chromosomal region. This discovery is intriguing because deletions and other rearrangements of this chromosomal region have been reported in a variety of tumor types, including malignant mesotheliomas and carcinomas of the lung, kidney, breast, and ovary (Kok et al., 1997). The existence of recurrent losses from 3 p implicates the involvement of a tumor suppressor gene(s) in this region.

### SUMMARY OF THE INVENTION

In accordance with one aspect of the present invention, novel oligonucleotide molecules are disclosed. The antisense molecules of the invention specifically hybridize with the nucleic acid molecule encoding human APPL and inhibit the expression human APPL. In a preferred embodiment, the antisense molecules of the invention are oligonucleotides comprising the sequences of SEQ ID NO: 1, SEQ ID NO: 2 and SEQ ID NO: 4. Also encompassed within the present invention are antisense analogs of SEQ ID NOS 1, 2, and 3 wherein the stability of the antisense molecules is enhanced by modifying the phosphodiester backbone of the molecules using phosphorothioates, 2′-O-methyloxymethyl ribonucleic acids or methylphosphonates.

In another embodiment of the invention, small, interfering RNA (siRNA) molecules are provided which also inhibit expression of APPL. An exemplary siRNA molecule has the sequence of SEQ ID NO: 6.

In still another embodiment of the invention, short, hairpin RNA (shRNA) molecules are provided which also inhibit expression of APPL. An exemplary shRNA molecule has the sequence of SEQ ID NO: 8. Furthermore, the shRNA may be expressed from a vector containing a promoter such as the polymerase III U6 promoter (see, e.g., Paddison and Hannon (2002) Cancer Cell, 2:17-23).

According to another aspect of the invention, a method is provided for controlling expression of APPL in human cells or tissues. The method comprises contacting human cells or tissues with the small nucleic acid molecules of the invention so that expression of APPL is altered. An exemplary method comprises providing an antisense molecule comprising the sequence of SEQ ID NO: 1, SEQ ID NO: 2 or SEQ ID NO: 4, which hybridizes to an expression-controlling sequence of a nucleic acid molecule encoding APPL. Upon administration in an amount effective to control APPL expression, the antisense oligonucleotide enters cells, binds specifically to an expression-controlling sequence of the nucleic acid molecule encoding APPL thereby inhibiting APPL expression. Alternatively, the siRNA of SEQ ID NO: 6 or shRNA of SEQ ID NO: 8 may be used in such a method. Alternatively, the small nucleic acid molecules of the invention (i.e., antisense oligonucleotides, siRNAs, and shRNAs) may be encoded for in a DNA vector which is brought into contact with the human cells or tissue. The siRNA and shRNA encoding vectors may contain the polymerase III U6 promoter

According to another aspect of the present invention, a method is provided for treating human malignancy due to the aberrant expression of an APPL protein. The method comprises delivering an antisense molecule comprising the sequence of SEQ ID NO: 1, SEQ ID NO: 2 or SEQ ID NO: 4 to a patient in need of such treatment. The method also encompasses delivery of an siRNA molecule of SEQ ID NO: 6 and an shRNA molecule of SEQ ID NO: 8.

In yet another embodiment of the invention, a pharmaceutical preparation is provided for treating human malignancy due to the aberrant expression of a APPL protein. The pharmaceutical preparation comprises an antisense oligonucleotide having the sequence of SEQ ID NO: 1, SEQ ID NO:2 or SEQ ID NO: 4, an siRNA of SEQ ID NO: 6, or an shRNA of SEQ ID NO: 8 in a biologically compatible medium. In another aspect, the pharmaceutical preparation further comprises at least one targeting agent for improving delivery of the antisense molecule to the cells expressing the aberrant protein. The pharmaceutical preparation may also contain at least one additional anti-cancer agent including without limitation, cisplatin, carboplatin, herceptin, taxol and taxane derivatives, cyclophosphamide, methotrexate, vincristin, etoposide and the like. Finally, the pharmaceutical preparation may comprise an analog of the antisense molecules of the invention.

### BRIEF DESCRIPTION OF THE DRAWINGS

FIG. 1 shows a graph illustrating inhibition of HeLa cell proliferation by various APPL antisense oligonucleotides.

FIG. 2 shows Western blots detecting APPL expression in HeLa cells in the presence of the control antisense oligonucleotide or antisense oligonucleotide #1 using the appropriate immunospecific antibody. Actin expression was measured as a control.

FIG. 3 shows FACS histograms of HeLa cells alone (top panels), HeLa cells treated with control oligonucleotide (middle panels) and HeLa cells treated with antisense oligonucleotide #1 (bottom panels).

FIG. 4 shows a three-dimensional graph illustrating the ratio of HeLa cells in G1, S and G2 cell cycle phases observed in the histograms presented in FIG. 3.

FIG. 5 shows a Western blot detecting APPL expression in HeLa cells during different phases of the cell cycle using the appropriate immunospecific antibody. Cyclin B1 and Survivin expression was also detected as a control.

FIGS. 6A-6D show micrographs of HeLa cells treated with either control oligonucleotides (FIGS. 6A and 6B) or antisense oligonucleotide #1 (FIGS. 6C and 6D). Cells were examined with Annexin V-Cy3 to detect phosphatidylserine on the cell surface. Morphological changes and cell death were observed in cells treated with antisense oligonucleotide #1 (FIG. 6D).

FIG. 7 shows a graph illustrating inhibition of HeLa cell proliferation by APPL siRNA.

FIG. 8 shows Western blots detecting APPL and Rac1 expression in HeLa cells in the presence of the control siRNA or APPL siRNA using the antibodies immunospecific for actin, Rac1 and APPL. Actin expression was measured as a control.

FIG. 9 shows a Western blot detecting APPL and β-actin expression in HeLa cells transfected with vector alone (Lane 1) or with a vector encoding shRNA (SEQ ID NO: 8; Lane 2). Proteins were detected with antibodies immunospecific for actin and APPL. Actin expression was measured as a control.

### DETAILED DESCRIPTION OF THE INVENTION

In accordance with the present invention, it has been discovered that APPL mRNA provides a suitable target for antisense therapy for the treatment of cancer. Targeting APPL mRNA with antisense molecules, siRNA, or shRNA molecules inhibits cell proliferation and/or induces cell death. Thus, targeted disruption of APPL expression has beneficial therapeutic implications for the treatment of human malignancy.

The detailed description set forth below describes preferred methods for practicing the present invention. Methods for selecting and preparing antisense oligonucleotides, siRNA molecules, shRNA molecules, and expression vectors encoding the small nucleic acids of the invention are provided, as well as methods for administering such compositions in vivo.

I. Definitions:

The following definitions are provided to facilitate an understanding of the present invention:

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):
Tₘ=81.5° C.+16.6Log[Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The term “probe” as used herein refers to an oligonucleotide, polynucleotide or DNA molecule, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to a DNA oligonucleotide, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The terms “percent similarity”, “percent identity” and “percent homology” when referring to a particular sequence are used as set forth in the University of Wisconsin GCG software program.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase small, interfering RNA (siRNA) refers to a double stranded RNA molecule (RNA is usually single stranded) which inhibits expression of its cognate mRNA (see, e.g. Ausubel et al., eds. Current Protocols in Molecular Biology, John Wiley and Sons, Inc., (1995)). A short hairpin RNA molecule (shRNA) typically consists of short inverted repeats separated by a small loop sequence. Generally, one of the inverted repeats is complimentary to the gene target. Additioanlly, the shRNA is typically processed into an siRNA within the cell by endonucleases. siRNAs and shRNAs specific for APPL and which downregulate its expression are exemplified herein.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

II. Selection and Preparation of Oligonucleotides:

Antisense oligonucleotides targeted to any known nucleotide sequence can be prepared by oligonucleotide synthesis according to standard methods. Synthesis of oligonucleotides via phosphoramidite chemistry is preferred, since it is an efficient method for preparing oligodeoxynucleotides, as well as being adapted to many commercial oligonucleotide synthesizers.

Selection of a suitable antisense sequence depends on knowledge of the nucleotide sequence of the target mRNA, or gene from which the mRNA is transcribed. In accordance with the present invention, antisense oligonucleotides #1 and #3 (described herein below; SEQ ID NOS: 1 and 3) target the translation initiation site of the nucleic acid molecule encoding APPL. Antisense oligonucleotide #3 specifically targets the junction between Exon 1 and Exon 2. In addition, antisense oligonucleotide #6 (SEQ ID NO: 4) specifically targets the sequence of Exon 3.

Similarly, selection of a suitable small interfering RNA (siRNA) molecule or a suitable shRNA molecule requires knowledge of the nucleotide sequence of the target mRNA, or gene from which the mRNA is transcribed. APPL siRNA (SEQ ID NO: 6) targets a region early within the transcript, specifically 83 bases 3′ from the start AUG.

Although targeting to mRNA is preferred and exemplified in the description below, it will be appreciated by those skilled in the art that other forms of nucleic acid, such as pre-mRNA or genomic DNA, may also be targeted.

Synthetic antisense oligonucleotides should be of sufficient length to hybridize to the target nucleotide sequence and exert the desired effect, i.e., blocking translation of an mRNA molecule. However, it should be noted that smaller oligonucleotides are likely to be more efficiently taken up by cells in vivo, such that a greater number of antisense oligonucleotides may be delivered to the location of the target mRNA. Preferably, antisense oligonucleotides should be at least 15 nucleotides long, to achieve adequate specificity. In a preferred embodiment of the invention, antisense molecules with 16 to 32 nucleotides in length are utilized. The siRNA molecules of the invention are typically between 12-30 nucleotides in length. Most preferably, siRNA molecules are about 21 nucleotides in length.

Small antisense oligonucleotides such as those described above are highly susceptible to degradation by assorted nucleases. Moreover, such molecules may be unable to enter cells because of insufficient membrane permeability. For these reasons, practitioners skilled in the art generally synthesize oligonucleotides that are modified in various ways to increase stability and membrane permeability. The use of modified antisense oligonucleotides is preferred in the present invention. The term “antisense oligonucleotide analog” refers to such modified oligonucleotides, as discussed hereinbelow.

Several methods of modifying oligodeoxyribo-nucleotides are known in the art. For example, methylphosphonate oligonucleotide analogs may be synthesized wherein the negative charge on the inter-nucleotide phosphate bridge is eliminated by replacing the negatively charged phosphate oxygen with a methyl group. See Uhlmann et al., Chemical Review, 90: 544-584 (1990). Another common modification, which is utilized in a preferred embodiment of the present invention, is the synthesis of oligodeoxyribonucleotide phosphorothioates. In these analogs, one of the phosphate oxygen atoms not involved in the phosphate bridge is replaced by a sulphur atom, resulting in the negative charge being distributed asymmetrically and located mainly on the sulphur atoms. When compared to unmodified oligonucleotides, oligonucleotide phosphorothioates are improved with respect to stability to nucleases, retention of solubility in water and stability to base-catalyzed hydrolysis. See Uhlmann et al., supra at 548-50; Cohen, J. S. (ed.) Oligodeoxynucleotides: Antisense Inhibitors of Gene Expression, CRC Press, Inc., Boca Raton, Fla. (1989).

Other modifications of oligodeoxyribonucleotides to produce stable, membrane permeable oligonucleotide analogs are commonly known in the art. For a review of such methods, see generally, Uhlmann et al., supra, and Cohen, supra which also describe methods for synthesis of such molecules. In addition, modified oligoribonucleotides may be utilized in the present invention. However, oligodeoxyribonucleotides are preferred due to their enhanced stability, ease of manufacture and the variety of methods available for analog synthesis.

Still other modifications of the oligonucleotides may include coupling sequences that code for RNase H to the antisense oligonucleotide. This enzyme (RNase H) will then hydrolyze the hybrid formed by the oligonucleotide and the specific targeted mRNA. Alkylating derivatives of oligonucleotides and derivatives containing lipophilic groups can also be used. Alkylating derivatives form covalent bonds with the mRNA, thereby inhibiting their ability to translate proteins. Lipophilic derivatives of oligonucleotides will increase their membrane permeability, thus enhancing penetration into tissue. Besides targeting the mRNAs, other antisense molecules can target the DNA, forming triple DNA helixes (DNA triplexes). Another strategy is to administer sense DNA strands which will bind to specific regulator cis or trans active protein elements on the DNA molecule.

Deoxynucleotide dithioates (phosphorodithioate DNA) may also be utilized in this invention. These compounds which have nucleoside-OPS₂O nucleoside linkages, are phosphorus achiral, anionic and are similar to natural DNA. They form duplexes with unmodified complementary DNA. They also activate RNase H and are resistant to nucleases, making them potentially useful as therapeutic agents. One such compound has been shown to inhibit HIV-1 reverse transcriptase (Caruthers et al., INSERM/NIH Conference on Antisense Oligonucleotides and Ribonuclease H, Arcachon, France 1992).

In accordance with the present invention, antisense RNA specific for APPL mRNA may be produced by expression of DNA sequences cloned into plasmid or retroviral vectors. Using standard methodology known to those skilled in the art, it is possible to maintain the antisense RNA-encoding DNA in any convenient cloning vector (see Ausubel et al., eds. Current Protocols in Molecular Biology, John Wiley and Sons, Inc., (1995)). In one embodiment, clones are maintained in a plasmid cloning/expression vector, such as pCEP4 (Invitrogen), which is propagated in a suitable host cell.

Similarly, the siRNA and shRNA constructs may be expressed in a vector by use of a DNA vector. Such vectors preferably contain RNA polymerase III promoters such as, without limitation, the U6 and H1 promoters (see, e.g., Myslinski et al. (2001) Nucl. Acids Res., 29:2502-09). In a particular method, the siRNA and shRNA constructs are expressed from the polymerase III U6 promoter.

Various genetic regulatory control elements may be incorporated into antisense RNA-encoding expression vectors to facilitate propagation in both eucaryotic and procaryotic cells. Different promoters may be utilized to drive expression of the APPL antisense sequences, the cytomegalovirus immediate early promoter being preferred as it promotes a high level of expression of downstream sequences. Polyadenylation signal sequences are also utilized to promote mRNA stability. Sequences preferred for use in the invention include, but are not limited to, bovine growth hormone polyadenylation signal sequences or thymidine kinase polyadenylation signal sequences. Antibiotic resistance markers are also included in these vectors to enable selection of transformed cells. These may include, for example, genes that confer hygromycin, neomycin or ampicillin resistance.

III. Administration of Oligonucleotides and/or Plasmid Vectors Producing RNA Molecules:

Anti-cancer oligonucleotides and/or RNA-encoding vectors as described herein are generally administered to a patient as a pharmaceutical preparation. The term “patient” as used herein refers to human or animal subjects.

The pharmaceutical preparation comprising the antisense oligonucleotides, shRNAs, siRNAs, or plasmid vectors encoding the small nucleic acids of the invention are conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of antisense oligonucleotides in the chosen medium will depend on the hydrophobic or hydrophilic nature of the medium, as well as the length and other properties of the antisense molecule. Solubility limits may be easily determined by one skilled in the art.

As used herein, “biologically acceptable medium” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the antisense molecules to be administered, its use in the pharmaceutical preparation is contemplated.

Selection of a suitable pharmaceutical preparation depends upon the method of administration chosen. For example, antisense oligonucleotides may be administered by direct injection into cancerous ovarian or pancreatic tissue. In this instance, a pharmaceutical preparation comprises the antisense molecule dispersed in a medium that is compatible with ovarian or pancreatic tissue.

Oligonucleotides antisense to APPL mRNAs, APPL siRNAs, or APPL shRNAs may be administered parenterally by intravenous injection into the blood stream, or by subcutaneous, intramuscular or intraperitoneal injection. Pharmaceutical preparations for parenteral injection are commonly known in the art. If parenteral injection is selected as a method for administering the antisense oligonucleotides, steps must be taken to ensure that sufficient amounts of the molecules reach their target cells to exert a biological effect. The lipophilicity of the antisense molecules, or the pharmaceutical preparation in which they are delivered may have to be increased so that the molecules can arrive at their target locations. Furthermore, the antisense molecules may have to be delivered in a cell-targeted carrier so that sufficient numbers of molecules will reach the target cells. Methods for increasing the lipophilicity of a molecule are known in the art, and include the addition of lipophilic groups to the antisense oligonucleotides. Two specific antisense carriers, phosphorothioate and methylphosphonate oligonucleotide analogs, become widely dispersed in living tissues following intravenous injection. For example, experiments in mice, which provided a detailed analysis of the pharmacokinetics, biodistribution and stability of oligonucleotide phosphorothioates showed a widespread distribution of phosphorothioate-modified oligodeoxynucleotides in most tissues for up to 48 hours. Significant amounts were found in brain following intraperitoneal or intravenous administration. Agrawal et al., Proc. Natl. Acad. Sci. USA, 88: 7595-99 (1991). In another study, methylphosphonate oligonucleotides were injected into mouse tail veins and found to achieve a reasonably uniform distribution in mouse tissue. See Uhlmann et al., supra at 577, citing Miller et al., Anti-Cancer Drug Design, 2: 117 (1987).

Several techniques have been used to increase the stability, cellular uptake and biodistribution of oligonucleotides. Antisense oligonucleotides of the present invention may be encapsulated in a lipophilic, targeted carrier, such as a liposome. One technique is to use as a carrier for the oligonucleotide a liposomal preparation containing the cationic lipid N-[1-(2,3-dioleyloxy)propyl]-N,N,N-trimethyl ammonium chloride (D OT MA; lipofectin). This has been found to increase by about 1000 fold the potency of the antisense oligonucleotide ISIS 1570, which hybridizes to the AUG translation initiation codon of human intracellular adhesion molecule-1. Bennett et al., Mol Pharmacol., 41: 1023-1033 (1992). Phosphorothioates have been particularly useful for increasing the biodistribution and stability of oligodeoxynucleotides in mice as described above. Loading phosphorothioate oligonucleotides into liposomes, particularly pH sensitive liposomes, to increase their cellular uptake has also been used with some success. Loke et al., Curr. Topics Microbiol. Immunol., 141: 282-289 (1988); Connor and Huang, Cancer Res., 46: 3431-3435 (1986).

Additional means by which antisense oligonucleotides may be administered include oral or rectal administration into the gastrointestinal tract, as well as intranasal or ophthalmic administration.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of antisense oligonucleotides directed to nucleic acids encoding APPL, APPL siRNAs, or APPL shRNAs may be determined by evaluating the toxicity of the antisense oligonucleotides in animal models. Various concentrations of antisense pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the antisense oligonucleotide treatment in combination with other standard anti-cancer drugs. The dosage units of antisense oligonucleotide may be determined individually in combination with each anti-cancer treatment according to greater shrinkage of tumors.

The pharmaceutical preparation comprising the antisense oligonucleotides may be administered at appropriate intervals, for example, twice a day until the pathological symptoms are reduced or alleviated, after which the dosage may be reduced to a maintenance level. The appropriate interval in a particular case would normally depend on the condition of the patient.

While the above discussion refers to the delivery of antisense oligonucleotides, it will be apparent to those skilled in the art that the methods described would also be suitable for the delivery of the vector constructs encoding APPL mRNA-specific antisense molecules, APPL siRNAs, or APPL shRNAs.

The following examples provide illustrative methods of practicing the instant invention, and are not intended to limit the scope of the invention in any way.

### EXAMPLE 1

### Inhibition of Hela Cell Proliferation by Appl Antisense Oligonucleotides

Oligonucleotides designed to target APPL mRNA were examined to determine whether they inhibit cellular proliferation. These oligonucleotides are provided in Table I:

TABLE I

Antisense Oligonucleotides

Anti-Oligonucleotide #1
5′ - TCCCCGGCATCGTGGCGG - 3′ (SEQ ID NO: 1)
Anti-Oligonucleotide #2
5′ - GACCTTGTCTGCGGGC - 3′ (SEQ ID NO: 2)
Anti-Oligonucleotide #3
5′ - GTGTGTTGCTGCACTTAATTC - 3′ (SEQ ID NO: 3)
Anti-Oligonucleotide #6
5′ - GGGCAGCTTGTCGATCCCCGGCATCGTGGCGG - 3′
(SEQ ID NO: 4)
Control Oligonucleotide
5′ - TGGGCGGCTACGTGCGCG - 3′ (SEQ ID NO: 5)

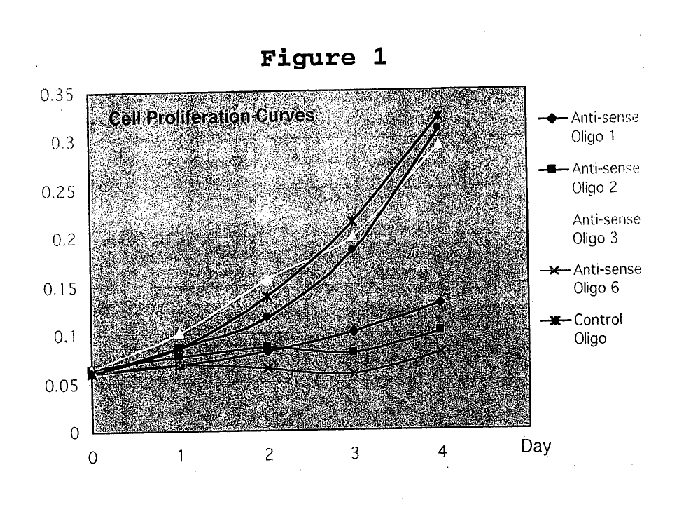

HeLa cells were incubated in culture medium containing 0.5 mM of antisense oligonucleotide #1, #2, #3, #6, or control oligonucleotide. PBS was also used as a non-transfection control medium. FIG. 1 shows the proliferation curves of HeLa cells incubated with the various antisense oligonucleotides. At the indicated day, cell numbers were determined by Cell Counting Kit (CCK-8; Alexis, Calif.). As illustrated by FIG. 1, antisense oligonucleotides #1, #2 and #6 dramatically reduced HeLa cell proliferation, while oligonucleotide #3 and the control oligonucleotide did not inhibit cellular growth (FIG. 1).

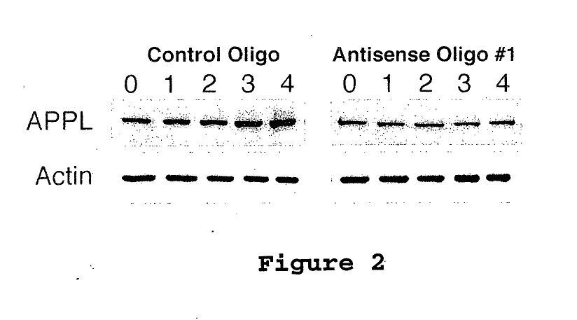

The lysates of cells treated with antisense oligonucleotide #1 or the control oligonucleotide were prepared at each time point and subjected to SDS-PAGE followed by Western blot analysis using anti-APPL antisera. Anti-actin antibody was used to evaluate equal loading on SDS-PAGE (FIG. 2). These data confirm that the oligonucleotides directed at APPL mRNA downregulated APPL expression.

### EXAMPLE 2

### Appl Antisense Oligonucleotide Modulation of Cell Cycle

To determine more precisely the inhibitive effects of APPL antisense oligonucleotides on cellular proliferation, the various phases of the cell cycle were monitored in HeLa cells treated with the antisense oligonucleotides.

Increased S Phase and Cell Death by APPL Antisense Oligonucleotide:

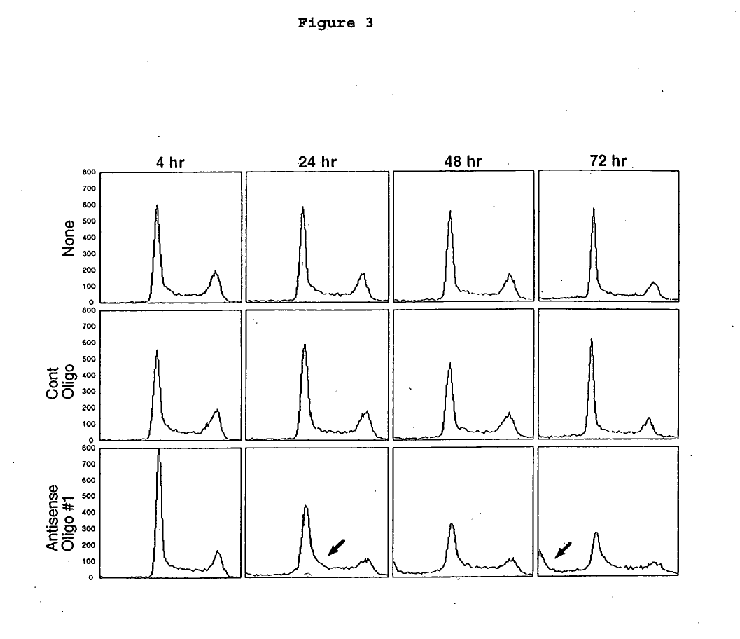

HeLa cells were treated with either antisense oligonucleotide #1 or the control oligonucleotide for 4, 24, 48, and 72 hours. At each time point, cells were harvested by trypsinization and centrifugation. The cells were then resuspended and washed with PBS buffer containing 0.5% FCS, followed by fixation with cold 70% ethanol for 60 minutes. The cells were washed with PBS+0.5% FCS, resuspended in PBS+0.5% FCS supplemented with Propidium Iodide and RNase A, and then incubated at 37° C. for 30 minutes. Flow cytometry analysis was performed using a FACSort flow cytometer, and the data were analyzed using the CellQuest software (Becton & Dickinson). The bottom panels of FIG. 3 shows the effects of APPL antisense oligonucleotide treatment on HeLa cells. As time increased, the number of viable cells detected decreased as a result of cell death and an increase in cells in S phase was observed.

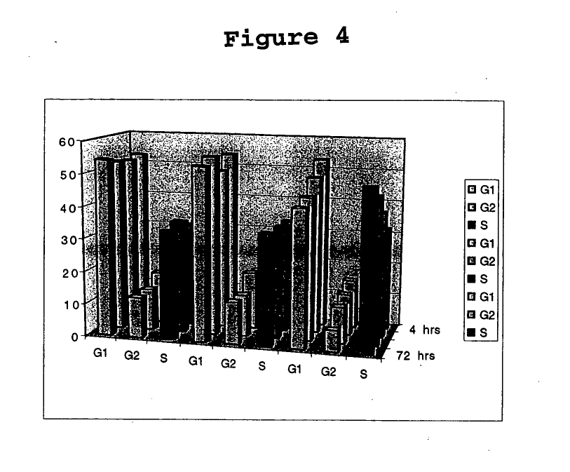

The ratio of HeLa cells in G1, S, and G2 cell cycle phases observed in each FACS analysis in FIG. 3 are graphically represented in FIG. 4.

Cell Cycle Analysis:

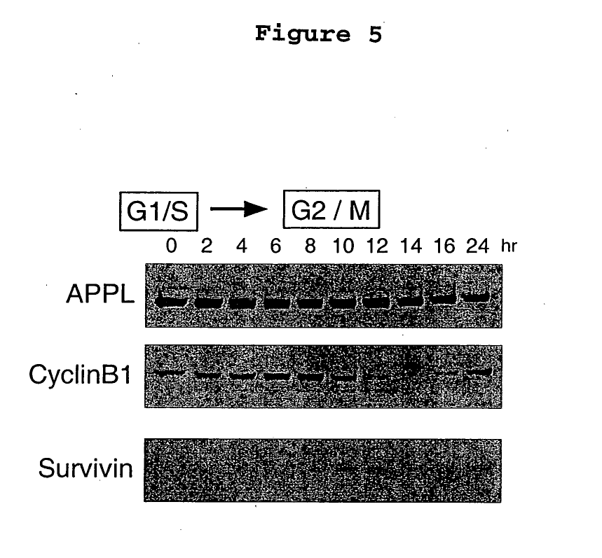

HeLa cells grown in DMEM supplemented with 10% FCS were synchronized at the G1/S phase in the cell cycle using a double thymidine block. The cells were treated with 2.5 mM thymidine (Sigma) for 16 hours to arrest the cell cycle, after which the thymidine was removed with three PBS washes, and the cell cycle was released using DMEM supplemented with 10% FCS for 10 hours. Thymidine (2.5 mM) was then added back to the cells and incubated for another 16 hours. Following release of the cell cycle, cell lysates were prepared at various time points and subjected to SDS-PAGE followed by Western blot analysis with anti-APPL antisera (FIG. 5). Antibodies of cyclin B1 and survivin were used to evaluate the phases of the cell cycle. The results indicated that APPL was expressed throughout the cell cycle.

### EXAMPLE 3

### Induction of Apoptosis using Appl Antisense Oligonucleotides

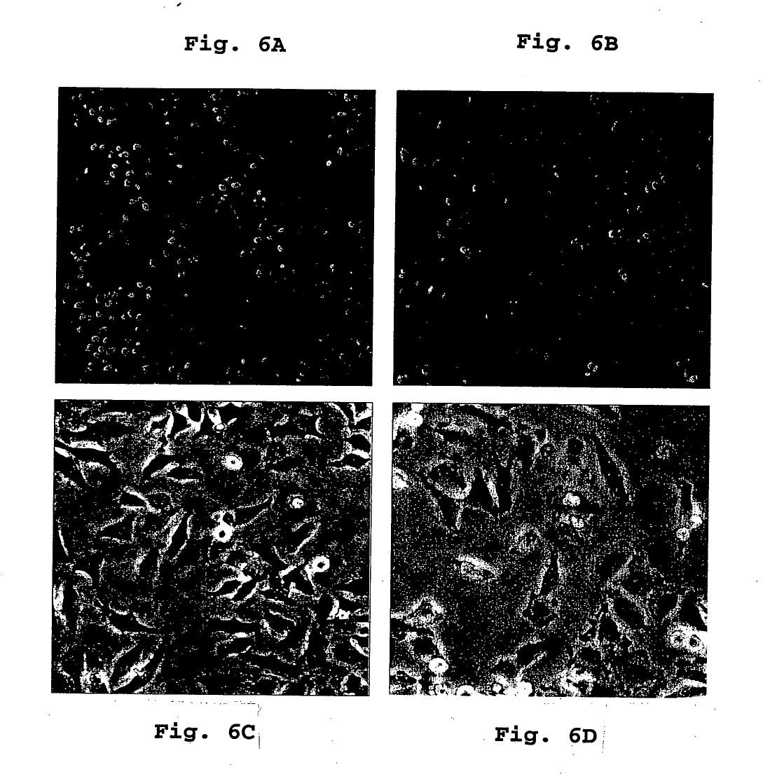

One potential outcome of down-regulation of APPL using APPL antisense oligonucleotides is the induction of apoptosis. Therefore, HeLa cells were examined for induction of apoptotic cell death during treatment with APPL antisense oligonucleotides. HeLa cells were treated with control oligonucleotide (FIGS. 6A and 6C) or antisense oligonucleotide #1 (FIG. 6B and 6D) for 48 hours. The cells were examined with Annexin V-Cy3 which detects phosphatidylserine on the cell surface, an indicator of the induction of apoptosis (FIGS. 6A and 6B). A comparison of cells treated with control oligonucleotide and antisense oligonucleotide #1 revealed morphological changes including suppression of filopodia and lamellopodia formation and cell motility as well as cell death in cells treated with antisense oligonucleotide #1 (FIGS. 6C and 6D).

These results indicate that APPL plays a significant role in cell proliferation, motility and invasiveness. Thus, targeted disruption of APPL expression provides a novel therapeutic strategy for the treatment of human malignancy.

### EXAMPLE 4

Inhibition of Hela Cell Proliferation by Appl siRNA

Small, interfering double stranded RNA molecules (siRNA) were designed to target APPL mRNA were examined to determine whether they inhibit cellular proliferation. These siRNAs are provided in Table II:

TABLE II

Oligonucleotides

APPL-siRNA
5′ - GAUGCCACAGCUAUUUCCAdTdT - 3′
(SEQ ID NO: 6)
3′ - dTdTCUACGGUGUCGAUAAAGGU
Control siRNA
5′ - CAGTCGCGTTTGCGACTGGTT - 3′ (SEQ ID NO: 7)

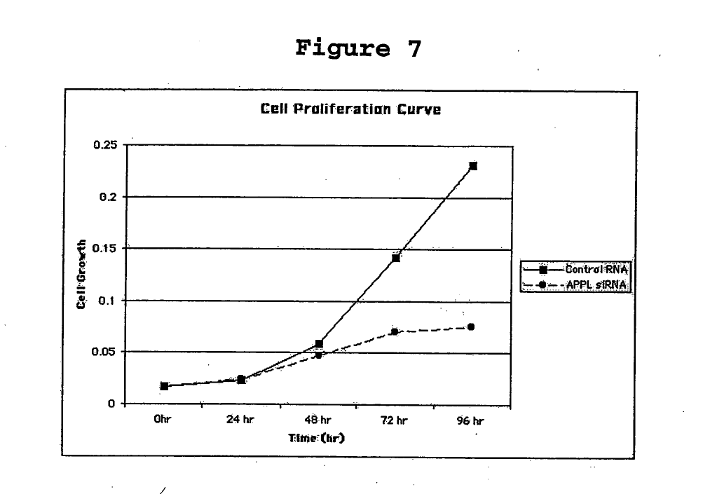

HeLa cells were transfected with 15 nM of APPL siRNA or control siRNA using GeneSilencer (Gene Therapy Systems, CA). FIG. 7 shows the proliferation curves of HeLa cells incubated with the various siRNAs. At the indicated day, cell numbers were determined by CCK-8 (Alexis). As illustrated by FIG. 7, APPL siRNA dramatically reduced HeLa cell proliferation, while the control siRNA did not inhibit cellular growth (FIG. 7).

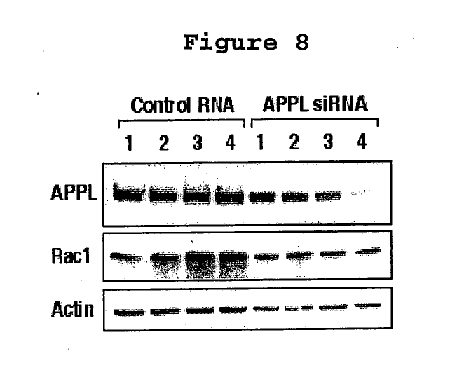

The lysates of cells treated with APPL siRNA or control siRNA were prepared at each time point and subjected to SDS-PAGE followed by Western blot analysis using anti-APPL antisera and anti-Rac1 antisera. Anti-actin antibody was used to evaluate equal loading on SDS-PAGE (FIG. 8). These data confirm that siRNAs silenced APPL mRNA expression. The data also show the concomitant downregulation of Rac1 expression.

### EXAMPLE 5

### Inhibition of Appl Expression by shRNA

A short, hairpin RNA molecule (shRNA) was designed which targets APPL mRNA and it is was determined whether this molecule inhibits APPL expression. This shRNA comprises the sequence:

(SEQ ID NO: 8)
5′ - AAAAAAGTCCACTGGAAGCAGCTACCAACCAACCTCAAGCTTCAA
GTCGGTTGATAGCTGCTTCCAGTAGACGGTGTTTCGTCCTTTCCACAA -
3′

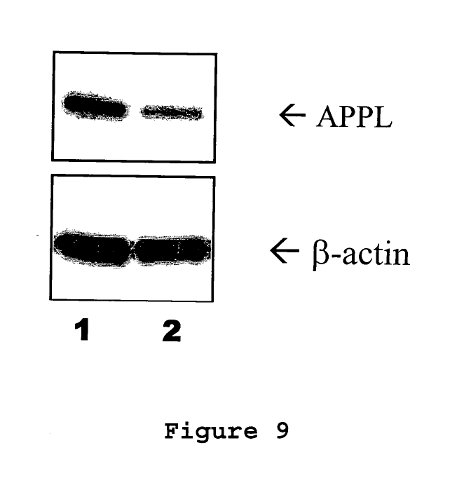

To test the ability of the shRNA to inhibit expression of APPL, the shRNA was cloned into the pPUR vector (BD Biosciences; Franklin Lakes, N.J.) and placed under the control of the human U6 promoter. HeLa cells were transiently transfected with vector alone or with vector containing the shRNA. 72 hours post-transfection, cellular lysates were prepared and subjected to SDS-PAGE followed by Western blot analysis using anti-APPL antisera and anti-actin antisera (FIG. 9). The anti-actin antibody was used to evaluate equal loading on SDS-PAGE. The decreased expression of APPL in cells transfected with the vector containing the shRHNA confirms that shRNAs silenced APPL mRNA expression.

### References

Altomare, D. A., Guo, K., Cheng, J. Q., Sonoda, G., Walsh, K. and Testa, J. R. (1995). Oncogene, 11, 1055-1060.
Altomare, D. A., Lyons, G. E., Mitsuuchi, Y., Cheng, J. Q. and Testa, J. R. (1998). Oncogene, 16, 2407-2411.
Blume-Jensen, P., Janknecht, R. and Hunter, T. (1998). Current Biol., 8, 779-782.
Calera, M. R., Martinez, C., Liu, H., Jack, A. K., Birnbaum, M. J. and Pilch, P. F. (1998). J. Biol. Chem., 273, 7201-7204.
Cheng, J. Q., Altomare, D. A., Klein, M. A., Lee, W. C., Kruh, G. D., Lissy, N. A. and Testa, J. R. (1997). Oncogene, 14, 2793-2801.
Cheng, J. Q., Godwin, A. K., Bellacosa, A., Taguchi, T., Franke, T. F., Hamilton, T. C., Tsichlis, P. N. and Testa, J. R. (1992). Proc. Natl. Acad. Sci. USA, 89, 9267-9271.
Cong, L. N., Chen, H., Li, Y., Zhou, L., McGibbon, M. A., Taylor, S. I. and Quon, M. J. (1997). Mol. Endocrinol., 11, 1881-1890.
Cross, D. A., Alessi, D. R., Cohen, P., Andjelkovich, M. and Hemmings, B. A. (1995). Nature, 378, 785-789.
Datta, S. R., Dudek, H., Tao, X., Masters, S., Fu, H., Gotoh, Y. and Greenberg, M. E. (1997). Cell, 91, 231-241.
del Peso, L., Gonzalez-Garcia, M., Page, C., Herrera, R. and Nunez, G. (1997). Science, 278, 687-689.
Delcommenne, M., Tan, C., Gray, V., Rue, L., Woodgett, J. and Dedhar, S. (1998). Proc. Natl. Acad. Sci. USA, 95, 4374-4379.
Jones, P. F., Jakubowicz, T. and Hemmings, B. A. (1991). Cell Regul., 2, 1001-1009.
Kavanaugh, W. M., Turck, C. W. and Williams, L. T. (1995) Science 268, 1177-1179.
Kok, K., Naylor, S. L. and Buys, C. H. C. M. (1997) Adv. Cancer. Res. 71, 27-92.
Liu, A. X., Testa, J. R., Hamilton, T. C., Jove, R., Nicosia, S. V. and Cheng, J. Q. (1998). Cancer Res., 58, 2973-2977.
Liu, Q. A. and Hengartner, M. O. (1998). Cell, 93, 961-972.
Mitsuuchi, Y., Johnson, S. W., Moonblatt, S. and Testa, J. R. (1998). J. Cell. Biochem., 70, 433-441.
Mitsuuchi, Y., Johnson, S. W., Moonblatt, S. and Testa, J. R. (1998). J. Cell. Biochem., 70, 433-441.
Ruggeri, B. A., Huang, L., Wood, M., Cheng, J. Q. and Testa, J. R. (1998). Mol. Carc., 21, 81-86.
Staal, S. P. (1987) Proc. Natl. Acad. Sci. USA 84, 5034-5037.
Walker, K. S., Deak, M., Paterson, A., Hudson, K., Cohen, P. and Alessi, D. R. (1998). Biochem. J., 331, 299-308.
Zhou, M. M., Ravichandran, K. S., Olejniczak, E. F., Petros, A. M., Meadows, R. P., Sattler, M., Harlan, J. E., Wade, W. S., Burakoff, S. J. and Fesik, S. W. (1995). Nature, 378, 584-592.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. An antisense molecule targeted to a nucleic acid molecule encoding human APPL, wherein said antisense molecule is complementary to an expression controlling sequence of said APPL encoding nuleic acid molecule, and upon binding to said APPL nucleic acid inhibits expression of APPL protein.

2. The antisense molecule of claim 1 which is an antisense oligonucleotide.

3. The antisense oligonucleotide of claim 2 selected from the group consisting of SEQ ID NO: 1, SEQ ID NO: 2 and SEQ ID NO: 4.

4. The antisense oligonucleotide of claim 3 having the sequence of SEQ ID NO:1, which is 5′-TCCCCGGCATCGTGGCGG-3′.

5. The antisense oligonucleotide of claim 3 having the sequence of SEQ ID NO: 2, which is 5′-GACCTTGTCTGCGGGC-3′.

6. The antisense oligonucleotide of claim 3 having the sequence of SEQ ID NO: 4, which is 5′-GGGCAGCTTGTCGATCCCCGGCATCGTGGCGG-3′.

7. The antisense oligonucleotide of claim 2, wherein said antisense oligonucleotide induces apoptotic cell death in human cells.

8. The antisense oligonucleotide of claim 2, wherein said antisense oligonucleotide comprises at least one modified internucleoside linkage.

9. The antisense oligonucleotide of claim 8, wherein said modified internucleoside linkage is a phosphorothioate linkage.

10. The antisense oligonucleotide of claim 2, wherein said antisense oligonucleotide comprises at least one modified sugar moiety.

11. The antisense oligonucleotide of claim 10, wherein said modified sugar moiety is a 2′-O-methyloxymethyl sugar moiety;

12. The antisense oligonucleotide of claim 2, wherein said antisense oligonucleotide comprises at least one modified base.

13. The antisense oligonucleotide of claim 12, wherein said modified base is a 5-methylcytosine.

14. A method of modulating the expression of APPL in human cells or tissues comprising contacting said cells or tissues with the antisense molecule of claim 1 in an amount sufficient to inhibit expression of APPL.

15. A method for controlling the expression of APPL in human cells, said method comprising:
a) providing an antisense oligonucleotide selected from the group consisting of SEQ ID NO: 1, SEQ ID NO: 2 or SEQ ID NO: 4, which hybridizes to an expression-controlling sequence of a nucleic acid molecule that encodes APPL; and
b) administering said antisense oligonucleotide to said humans cells in an amount sufficient to control expression of said APPL, whereby said antisense oligonucleotide enters said cells expressing APPL and binds specifically to the expression-controlling sequence of said nucleic acid molecule encoding APPL thereby inhibiting APPL expression.

16. The method according to claim 15, wherein said human cells are selected from the group consisting of skeletal muscle, heart, ovary, and pancreas.

17. The method according to claim 15, wherein administration of said antisense oligonucleotide induces apoptotic cell death in said human cell.

18. The method according to claim 15, wherein said antisense oligonucleotide is an antisense oligonucleotide analog.

19. The antisense oligonucleotide of claim 2, wherein said antisense oligonucleotide is encoded by DNA.

20. A vector comprising the DNA which encodes the antisense oligonucleotide of claim 19.

21. A method of treatment for human malignancy due to the expression of an aberrant APPL protein in a patient requiring such treatment, said method comprising delivery of an antisense oligonucleotide of claim 3 in an amount sufficient to control expression of said protein.

22. The method of claim 21, further comprising administration of at least one additional anti-cancer agent selected from the group consisting of cisplatin, carboplatin, herceptin, taxol, taxane derivatives, cyclophosphamide, methotrexate, vincristin, and etoposide.

23. A pharmaceutical preparation for treating human malignancy due to the expression by cells of an aberrant APPL protein, comprising an antisense APPL oligonucleotide in a biologically compatible medium.

24. A pharmaceutical preparation according to claim 23, wherein said antisense oligonucleotide comprises the sequence of SEQ ID NO:1, which is 5′-TCCCCGGCATCGTGGCGG-3′.

25. A pharmaceutical preparation according to claim 23, wherein said antisense oligonucleotide comprises the sequence of SEQ ID NO:2, which is 5′-GACCTTGTCTGCGGGC-3′.

26. A pharmaceutical preparation according to claim 23, wherein said antisense oligonucleotide comprises the sequence of SEQ ID NO:4, which is 5′-GGGCAGCTTGTCGATCCCCGGCATCGTGGCGG -3′.

27. A pharmaceutical preparation according to claim 23, which further comprises at least one targeting agent for improving delivery of said antisense oligonucleotide to said cells expressing said protein.

28. A pharmaceutical preparation according to claim 27, wherein said at least one targeting agent comprises a lipid.

29. A pharmaceutical preparation according to claim 23, wherein said antisense oligonucleotide is encapsulated in a lipid vesicle.

30. The pharmaceutical preparation according to claim 23, wherein said antisense oligonucleotide is selected from the group consisting of SEQ ID NO: 1, SEQ ID NO: 2 and SEQ ID NO: 4, and is an analog having a modified internucleotide linkage.

31. The pharmaceutical preparation of claim 30, further comprising at least one additional anti cancer agent.

32. The pharmaceutical preparation of claim 31, wherein said anti-cancer agent is selected from the group consisting of cisplatin, carboplatin, herceptin, taxol, taxane derivatives, cyclophosphamide, methotrexate, vincristin, and etoposide.

33. The pharmaceutical preparation of claim 23, wherein said antisense APPL oligonucleotide is encoded in a vector.

34. A double stranded RNA molecule targeted to a nucleic acid molecule encoding human APPL, wherein said double stranded RNA molecule inhibits expression of APPL protein upon entry into a cell comprising APPL encoding nucleic acids.

35. The double stranded RNA molecule of claim 34, wherein said double stranded RNA molecule is an siRNA.

36. The double stranded RNA molecule of claim 34, wherein said double stranded RNA molecule is an shRNA.

37. An siRNA molecule as claimed in claim 35, having the sequence of SEQ ID NO: 6.

38. An shRNA molecule as claimed in claim 36, having the sequence of SEQ ID NO: 8.

39. A method of modulating the expression of APPL in human cells or tissues comprising contacting said cells or tissues with the siRNA molecule of claim 35 in an amount sufficient to inhibit expression of APPL.

40. A method of modulating the expression of APPL in human cells or tissues comprising contacting said cells or tissues with the shRNA molecule of claim 36 in an amount sufficient to inhibit expression of APPL.

41. A method for controlling the expression of APPL in human cells, said method comprising:
a) providing a siRNA molecule of SEQ ID NO: 6; and
b) administering said siRNA molecule to said humans cells in an amount sufficient to control expression of said APPL, whereby said siRNA molecule enters said cells expressing APPL and inhibits APPL expression.

42. The method according to claim 41, wherein said human cells are selected from the group consisting of skeletal muscle, heart, ovary, and pancreas.

43. A method for controlling the expression of APPL in human cells, said method comprising:
a) providing a shRNA molecule of SEQ ID NO: 8; and
b) administering said siRNA molecule to said humans cells in an amount sufficient to control expression of said APPL, whereby said siRNA molecule enters said cells expressing APPL and inhibits APPL expression.

44. The method according to claim 43, wherein said human cells are selected from the group consisting of skeletal muscle, heart, ovary, and pancreas.

45. A method for controlling the expression of APPL in human cells, said method comprising:
a) providing a vector encoding an siRNA molecule of SEQ ID NO: 6; and
b) administering said vector to said humans cells in an amount sufficient to control expression of said APPL, whereby said vector enters said cells expressing APPL and inhibits APPL expression.

46. The method according to claim 45, wherein said human cells are selected from the group consisting of skeletal muscle, heart, ovary, and pancreas.

47. A method for controlling the expression of APPL in human cells, said method comprising:
a) providing a vector encoding an shRNA molecule of SEQ ID NO: 8; and
b) administering said vector to said humans cells in an amount sufficient to control expression of said APPL, whereby said vector enters said cells expressing APPL and inhibits APPL expression.

48. The method according to claim 47, wherein said human cells are selected from the group consisting of skeletal muscle, heart, ovary, and pancreas.

49. A method of treatment for human malignancy due to the expression of an aberrant APPL protein in a patient requiring such treatment, said method comprising delivery of a siRNA molecule of claim 35 in an amount sufficient to control expression of said protein.

50. The method of claim 49, further comprising administration of at least one additional anti-cancer agent selected from the group consisting of cisplatin, carboplatin, herceptin, taxol, taxane derivatives, cyclophosphamide, methotrexate, vincristin, and etoposide.

51. A method of treatment for human malignancy due to the expression of an aberrant APPL protein in a patient requiring such treatment, said method comprising delivery of a shRNA molecule of claim 36 in an amount sufficient to control expression of said protein.

52. The method of claim 51, further comprising administration of at least one additional anti-cancer agent selected from the group consisting of cisplatin, carboplatin, herceptin, taxol, taxane derivatives, cyclophosphamide, methotrexate, vincristin, and etoposide.

53. A pharmaceutical preparation for treating human malignancy due to the expression by cells of an aberrant APPL protein, comprising an APPL siRNA in a biologically compatible medium.

54. The pharmaceutical preparation according to claim 53, wherein said siRNA comprises the sequence of SEQ ID NO:6.

55. The pharmaceutical preparation according to claim 54, which further comprises at least one targeting agent for improving delivery of said siRNA to said cells expressing said protein.

56. The pharmaceutical preparation according to claim 55, wherein said at least one targeting agent comprises a lipid.

57. The pharmaceutical preparation according to claim 56, wherein said siRNA is encapsulated in a lipid vesicle.

58. The pharmaceutical preparation of claim 54, further comprising at least one additional anti cancer agent.

59. The pharmaceutical preparation of claim 58, wherein said anti-cancer agent is selected from the group consisting of cisplatin, carboplatin, herceptin, taxol, taxane derivatives, cyclophosphamide, methotrexate, vincristin, and etoposide.

60. The pharmaceutical preparation of claim 53, wherein said siRNA is encoded in a vector.

61. A pharmaceutical preparation for treating human malignancy due to the expression by cells of an aberrant APPL protein, comprising an APPL shRNA in a biologically compatible medium.

62. The pharmaceutical preparation according to claim 61, wherein said shRNA comprises the sequence of SEQ ID NO: 8.

63. The pharmaceutical preparation according to claim 62, which further comprises at least one targeting agent for improving delivery of said shRNA to said cells expressing said protein.

64. The pharmaceutical preparation according to claim 63, wherein said at least one targeting agent comprises a lipid.

65. The pharmaceutical preparation according to claim 64, wherein said shRNA is encapsulated in a lipid vesicle.

66. The pharmaceutical preparation of claim 61, further comprising at least one additional anti cancer agent.

67. The pharmaceutical preparation of claim 66, wherein said anti-cancer agent is selected from the group consisting of cisplatin, carboplatin, herceptin, taxol, taxane derivatives, cyclophosphamide, methotrexate, vincristin, and etoposide.

68. The pharmaceutical preparation of claim 61, wherein said shRNA is encoded in a vector.
